(19) United States
(12) Patent Application Publication
(10) Pub. No.: US 2002/0051821 A1
(43) Pub. Date: May 2, 2002

(54) Aliginate particle formulation

(76) Inventors: Sung-Yun Kwon, Fremont, CA (US); Frank Kochinke, Fremont, CA (US)

Correspondence Address: Thomas P. McCracken, POWDERJECT TECHNOLOGIES INC., 6511 Dumbarton Circle, Fremont, CA, 94555, US

(21) Appl. No.: 09/949,392

(22) Filed: Sep. 7, 2001

Related U.S. Application Data
(60) Provisional application No. 60/231,119, filed on Sep. 8, 2000.

(51) Int. Cl. A61K 39/00; A61K 39/38; A61K 9/14

(52) U.S. Cl. 424/489; 424/184.1

(57) ABSTRACT

Gelled alginate particles suitable for administration by needleless injection are loaded with a pharmacologically active agent and have a mean mass aerodynamic diameter of from 0.1 to 250 μm and an envelope density of from 0.1 to 2.5 g/cm³.

### CROSS-REFERENCE TO RELATED APPLICATION

This application is related to U.S. provisional application Ser. No. 60/231,119, filed Sep. 8, 2000, from which priority is claimed pursuant to 35 U.S.C. §119(e)(1) and which application is incorporated herein by reference in its entirety.

### TECHNICAL FIELD

The present invention relates to particulate compositions that are suitable for transdermal particle delivery from a needleless syringe system. More particularly, the present invention relates to particulate compositions formed using an alginate material, and methods of forming those compositions.

### BACKGROUND OF THE INVENTION

The ability to deliver pharmaceutical agents into and through skin surfaces (transdermal delivery) provides many advantages over oral or parenteral delivery techniques. In particular, transdermal delivery provides a safe, convenient and noninvasive alternative to traditional administration systems, conveniently avoiding the major problems associated with oral delivery (e.g. variable rates of absorption, gastric degradation and metabolism, hepatic first pass effect, gastrointestinal irritation and/or bitter or unpleasant drug tastes) or parenteral delivery (e.g. needle pain, the risk of introducing infection to treated individuals, the risk of contamination or infection of health care workers caused by accidental needle-sticks and the disposal of used needles).

However, despite its clear advantages, transdermal delivery presents a number of its own inherent logistical problems. Passive delivery through intact skin necessarily entails the transport of molecules through a number of structurally different tissues, including the stratum corneum (the major barrier), the viable epidermis, the papillary dermis and the capillary walls in order for the drug to gain entry into the blood or lymph system. Transdermal delivery systems must therefore be able to overcome the various resistances presented by each type of tissue.

In light of the above, a number of alternatives to passive transdermal delivery have been developed. These alternatives include the use of skin penetration enhancing agents, or “permeation enhancers”, to increase skin permeability, as well as non-chemical modes such as the use of iontophoresis, electroporation or ultrasound. However, these alternative techniques often give rise to their own unique side effects such as skin irritation or sensitization. Thus, the spectrum of agents that can be safely and effectively administered using traditional transdermal delivery methods has remained limited.

More recently, a novel transdermal drug delivery system that entails the use of a needleless syringe to fire powders (i.e. solid drug-containing particles) in controlled doses into and through intact skin has been described. In particular, commonly owned U.S. Pat. No. 5,630,796 to Bellhouse et al describes a needleless syringe that delivers pharmaceutical particles entrained in a transiently supersonic gas flow. The needleless syringe is used for transdermal delivery of powdered drug compounds and compositions, for delivery of genetic material into living cells (e.g. gene therapy) and for the delivery of biopharmaceuticals into skin and, via blood or lymph, to other tissues via the systemic ciruclation. The needleless syringe can also be used in conjunction with surgery to deliver drugs and biologics to organ surfaces, solid tumors and/or to surgical cavities (e.g. tumor beds or cavities after tumor resection). In theory, practically any pharmaceutical agent that can be prepared in a substantially solid, particulate form can be safely and easily delivered using such devices.

The physical characteristics of the particles do however need to be engineered to meet the particular demands of administration via a needleless syringe. It is important that particles for delivery via a needleless syringe have a structural integrity such that they can survive the action of the gas jet of the syringe and the ballistic impact with skin or mucosal tissue at high velocities. It is also important that the particles have a density that enables the particles to achieve sufficient momentum to penetrate tissue. Particles are typically fired at very high velocities from a needleless syringe, accelerated by gas flows briefly at supersonic velocities such as from Mach 1 to Mach 8.

### SUMMARY OF THE INVENTION

This invention is based on the discovery that pharmacologically active agents can be loaded within gelled alginate particles and successfully delivered to a subject, particularly a human, by transdermal particle injection. The particles have sufficient structural integrity to withstand being fired from a needleless syringe, and impacting and penetrating skin or mucosal tissue at high velocity. A high loading of the active agent and a narrow particle size distribution can be achieved.

Accordingly, the present invention provides a method of delivering a pharmacologically active agent to a subject, which method comprises the step of administering to the subject by needleless injection an effective amount of alginate particles which are loaded with the said agent and which have a mean mass aerodynamic diameter of from 0.1 to 250 μm and an envelope density of from 0.1 to 2.5 g/cm³.

The invention also provides:

a dosage receptacle for a needleless syringe, said receptacle containing alginate particles which are loaded with a pharmacologically active agent and which have a mean mass aerodynamic diameter of from 0.1 to 250 μm and an envelope density of from 0.1 to 2.5 g/cm³;

alginate particles suitable for administration to a subject by needleless injection, wherein the particles are loaded with a pharmacologically active agent, the mean mass aerodynamic diameter of the particles is from 10 to 70 μm, less than 10% by weight of the particles have a diameter which is at least 5 μm greater or at least 5 μm less than the mean mass aerodynamic diameter, the envelope density of the particles is from 0.8 to 1.5 g/cm³, and the ratio of the major axis: minor axis of the particles is 3:1 to 1:1;

a process for the preparation of alginate particles suitable for administration to a subject by needleless injection wherein the particles are loaded with a pharmacologically active agent, the mean mass aerodynamic diameter of the particles is from 10 to 70 μm, less than 10% by weight of the particles have a diameter which is at least 5 μm greater or at least 5 μm less than the mean mass aerodynamic diameter, the envelope density of the particles is from 0.8 to 1.5 g/cm³, and the ratio of the major axis:minor axis of the particles is 3:1 to 1:1; which process comprises the steps of:

(a) providing an aqueous solution or dispersion of the pharmacologically active agent, within which solution or dispersion a water-soluble alginate is dissolved;

(b) mixing the aqueous solution or dispersion with a sufficient amount of a water-immiscible solvent so as to form an emulsion in which droplets of the aqueous solution or dispersion are dispersed in the water-immiscible solvent;

(c) adding a divalent or trivalent metal cation which gels the alginate;

(d) collecting the resultant gelled alginate particles loaded with the pharmacologically active agent; and

(e) if necessary, separating therefrom the required alginate particles;

a process for the preparation of alginate particles suitable for use in a needleless injection, wherein the particles are loaded with a pharmacologically active agent, the mean mass aerodynamic diameter of the particles is from 10 to 100 μm, less than 10% by weight of the particles have a diameter which is at least 5 μm greater or at least 5 μm less than the mean mass aerodynamic diameter, the envelope density of the particles is from 0.8 to 1.5 g/cm³, and the ratio of the major axis:minor axis of the particles is 3:1 to 1:1; which process comprises the steps of:

(a) providing pre-formed alginate particles which are not loaded with the pharmacologically active agent;

(b) contacting the particles with an aqueous solution or dispersion of the pharmacologically active agent for a period of time sufficient to allow the particles to swell and incorporate the active agent therewithin; and

(c) collecting the particles thus loaded with the pharmacologically active agent;

(d) if necessary, separating therefrom the required alginate particles; and

use of a pharmacologically active agent in the manufacture of a medicament for needleless injection comprising alginate particles which are loaded with the pharmacologically active agent and which have mean mass aerodynamic diameter of from 0.1 to 250 μm and an envelope density of from 0.1 to 2.5 g/cm³.

In one embodiment, the alginate particles are loaded with an expressible gene construct encoding an antigen. The invention can thus be used for nucleic acid vaccination. In another embodiment, the particles are loaded with an antigen and can be delivered as a vaccine by particle injection.

It is an advantage of the present invention that the alginate particles can be used as carrier systems for pharmacologically active agents, thereby facilitating high-velocity particle injection delivery performance of such agents. Since release of the active agent will typically be dependent upon a number of factors, such as the degree of swelling experienced by the alginate particles when delivered to an aqueous environment; dissolution of a crystallized active agent from the alginate particles; diffusion of the active agent from the alginate matrix; and degradation of the alginate matrix; numerous delivery profiles can be readily tailored for each active agent.

These and other objects, aspects, embodiments and advantages of the present invention will readily occur to those of ordinary skill in the art in view of the disclosure herein.

### BRIEF DESCRIPTION OF THE FIGURES

FIG. 1 shows the modified Franz cell used in Example 1.

FIG. 2 is an axial section through a unit dosage receptacle containing a dose of the alginate particles of the present invention.

### DETAILED DESCRIPTION OF THE PREFERRED EMBODIMENTS

Before describing the present invention in detail, it is to be understood that this invention is not limited to particularly exemplified alginate powder formulations or process parameters as such may, of course, vary. It is also to be understood that the terminology used herein is for the purpose of describing particular embodiments of the invention only, and is not intended to be limiting.

All publications, patents and patent applications cited herein, whether supra or infra, are hereby incorporated by reference in their entirety.

It must be noted that, as used in this specification and the appended claims, the singular forms “a,” “an” and “the” include plural referents unless the content clearly dictates otherwise. Thus, for example, reference to “a particle” includes a mixture of two or more such particles, reference to “a pharmaceutical” includes mixtures of two or more such agents, and the like.

A. Definitions

Unless defined otherwise, all technical and scientific terms used herein have the same meaning as commonly understood by one of ordinary skill in the art to which the invention pertains. The following terms are intended to be defined as indicated below.

The term “powder” as used herein refers to a composition that consists of substantially solid particles that can be delivered transdermally using a needleless syringe device. The particles that make up the powder can be characterized on the basis of a number of parameters including, but not limited to, average particle size, average particle density, particle morphology (e.g. particle aerodynamic shape and particle surface characteristics) and particle penetration energy (P.E.).

The average particle size of the powders according to the present invention can vary widely and will generally range from about 0.1 to about 250 μm, for example from about 10 to about 100 μm and more typically from about 20 to about 70 μm. The average particle size of the powder can be measured as a mass mean aerodynamic diameter (MMAD) using conventional techniques such as microscopic techniques (where particles are sized directly and individually rather than grouped statistically), absorption of gases, permeability, light obscuration or time of flight. If desired, automatic particle-size counters can be used (e.g. Aerosizer, Coulter Counter, HIAC Counter, or Gelman Automatic Particle Counter) to ascertain the average particle size.

Actual particle density or “absolute density” can be readily ascertained using known quantification techniques such as helium pycnometry and the like. Alternatively, envelope (“tap”) density measurements can be used to assess the density of a powder according to the invention. The envelope density of a powder of the invention will generally range from about 0.1 to 2.5 g/cm³, preferably from about 0.8 to about 1.5 g/cm³.

Envelope density information is particularly useful in characterizing the density of objects of irregular size and shape. Envelope density is the mass of an object divided by its volume, where the volume includes that of its pores and small cavities but excludes interstitial space. A number of methods of determining envelope density are known in the art, including wax immersion, mercury displacement, water absorption and apparent specific gravity techniques. A number of suitable devices are also available for determining envelope density, for example, the Geopyc™ Model 1360, available from the Micromeritics Instrument Corp. The difference between the absolute density and envelope density of a sample pharmaceutical composition provides information about the sample's percentage total porosity and specific pore volume.

Particle morphology, particularly the aerodynamic shape of a particle, can be readily assessed using standard light microscopy. It is preferred that the particles have a substantially spherical or at least substantially elliptical aerodynamic shape. It is also preferred that the particles have an axis ratio of 3 or less to avoid the presence of rod- or needle-shaped particles. These same microscopic techniques can also be used to assess the particle surface characteristics, e.g. the amount and extent of surface voids, irregularities or roughness, or degree of porosity.

Particle penetration energies can be ascertained using a number of conventional techniques, for example a metallized film P.E. test. A metallized film material (e.g. a 125 μm polyester film having a 350 Å layer of aluminum deposited on a single side) is used as a substrate into which the powder is fired from a needleless syringe (e.g. the needleless syringe described in U.S. Pat. No. 5,630,796 to Bellhouse et al.) at an initial velocity of about 100 to 3000 m/sec. The metallized film is placed, with the metal coated side facing upwards, on a suitable surface.

A needleless syringe loaded with a powder is placed with its spacer contacting the film, and then fired. Residual powder is removed from the metallized film surface using a suitable solvent. Penetration energy is then assessed using a BioRad Model GS-700 imaging densitometer to scan the metallized film, and a personal computer with a SCSI interface and loaded with MultiAnalyst software (BioRad) and Matlab software (Release 5.1, The MathWorks, Inc.) is used to assess the densitometer reading. A program is used to process the densitometer scans made using either the transmittance or reflectance method of the densitometer. The penetration energy of the powders should be equivalent to, or better than that of reprocessed mannitol particles of the same size (mannitol particles that are freeze-dried, compressed, ground and sieved according to the methods of commonly owned International Publication No. WO 97/48485, incorporated herein by reference).

The term “subject” refers to any member of the subphylum cordata including, without limitation, humans and other primates including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The methods described herein are intended for use in any of the above vertebrate species, since the immune systems of all of these vertebrates operate similarly.

The term “transdermal delivery” includes both transdermal (“percutaneous”) and transmucosal routes of administration, i.e. delivery by passage through the skin or mucosal tissue. See, e.g., Transdermal Drug Delivery: Developmental Issues and Research Initiatives, Hadgraft and Guy (eds.), Marcel Dekker, Inc., (1989); Controlled Drug Delivery: Fundamentals and Applications, Robinson and Lee (eds.), Marcel Dekker Inc., (1987); and Transdermal Delivery of Drugs, Vols. 1-3, Kydonieus and Berner (eds.), CRC Press, (1987).

B. General Methods

The invention is concerned with delivering a pharmacologically active agent to a subject by particle delivery. The particles comprise an alginate matrix within which the active agent is held. The particles have a size and density suitable for needleless injection. They can withstand ballistic impact with the target skin, tissue or mucosal surface upon delivery from a needleless syringe. The particles are typically provided as a free-flowing powder that may be produced in bulk, transported in containers or prepared as a unit dosage for use with a needleless syringe.

In the present invention, the alginate particles are gel particles. Alginate gels in the presence of divalent and trivalent metal cations other than Mg²⁺. The particles thus incorporate such a gel-forming divalent or trivalent metal cation. The cation may be a cation selected from Ca²⁺, Ba²⁺, Sr²⁺, Zn²⁺ or Al³⁺. Typically the cation is Ca^{2 +}, optionally with a second cation such as Ba²⁺, Sr²⁺, Zn²⁺ or Al³⁺. Preferably, therefore, the alginate is a calcium alginate.

Alginate is a linear polymer which has 1,4′-linked β-D-mannuronic acid (mannuronate) and α-L-guluronic acid (guluronate) residues arranged as blocks or randomly. The properties of the alginate are influenced by the content of the mannuronate and guluronate residues. The alginate may have 60 to 70 wt % guluronate residues and 40 to 30 wt % mannuronate residues. Such an alginate is termed “a high G alginate.” The alginate may be “a low G alginate,” that is, of 25 to 35 wt % guluronate residues and 75 to 65 wt % mannuronate residues. Alternatively, the alginate can be “a medium G alginate” having 35 to 60 wt % guluronate residues and 65 to 40 wt % mannuronate residues.

The pharmacologically active agent with which the alginate particles are loaded includes any substance which, when administered to a human or animal subject, induces a desired pharmacologic and/or physiologic effect by local and/or systemic action. The term therefore encompasses those compounds or chemicals traditionally regarded as drugs, biopharmaceuticals (including molecules such as peptides, proteins and nucleic acids), vaccines and genes.

Pharmacologically active agents useful in the invention include drugs acting at synaptic and neuroeffector junctional sites (cholinergic agonists, anticholinesterase agents, atropine, scopolamine, and related antimuscarinic drugs, catecholamines and sympathomimetic drugs, and adrenergic receptor antagonists); drugs acting on the central nervous systems; autacoids (drug therapy of inflammation); drugs affecting renal function and electrolyte metabolism; cardiovascular drugs; drugs affecting gastrointestinal function; chemotherapy of neoplastic diseases; drugs acting on the blood and the blood-forming organs; and hormones and hormone antagonists. Thus, the agents include, but are not limited to anti-infectives such as antibiotics and antiviral agents; analgesics and analgesic combinations; local and general anesthetics; anorexics; antiarthritics; antiasthmatic agents; anticonvulsants; antidepressants; antihistamines; anti-inflammatory agents; antinauseants; antimigrane agents; antineoplastics; antipruritics; antipsychotics; antipyretics; antispasmodics; cardiovascular preparations (including calcium channel blockers, beta-blockers, beta-agonists and antiarrythmics); antihypertensives; diuretics; vasodilators; central nervous system stimulants; cough and cold preparations; decongestants; diagnostics; hormones; bone growth stimulants and bone resorption inhibitors; enzymes; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including double- and single-stranded molecules and supercoiled or condensed molecules, gene constructs, expression vectors, plasmids, antisense molecules and the like.

Specific examples of drugs useful in this invention include angiotensin converting enzyme (ACE) inhibitors, β-lactam antibiotics and γ-aminobutyric acid (GABA)-like compounds. Representative ACE inhibitors are discussed in Goodman and Gilman, Eighth Edition at pp. 757-762, which is incorporated herein by reference. These include quinapril, ramipril, captopril, benzepril, fosinopril, lisinopril, enalapril, and the like and the respective pharmaceutically acceptable salts thereof. Beta-lactam antibiotics are those characterized generally by the presence of a beta-lactam ring in the structure of the antibiotic substance and are discussed in Goodman and Gilman, Eighth Edition at pp. 1065 to 1097, which is incorporated herein by reference. These include penicillin and its derivatives such as amoxicillin and cephalosporins. GABA-like compounds may also be found in Goodman and Gilman. Other compounds include calcium channel blockers (e.g., verapamil, nifedipine, nicardipine, nimodipine and diltiazem); bronchodilators such as theophylline; appetite suppressants, such as phenylpropanolamine hydrochloride; antitussives, such as dextromethorphan and its hydrobromide, noscapine, carbetapentane citrate, and chlophedianol hydrochloride; antihistamines, such as terfenadine, phenidamine tartrate, pyrilamine maleate, doxylamine succinate, and phenyltoloxamine citrate; decongestants, such as phenylephrine hydrochloride, phenylpropanolamine hydrochloride, pseudoephedrine hydrochloride, chlorpheniramine hydrochlordie, pseudoephedrine hydrochloride, chlorpheniramine maleate, ephedrine, phenylephrine, chlorpheniramine, pyrilamine, phenylpropanolamine, dexchlorpheniramine, phenyltoxamine, phenindamine, oxymetazoline, methscopalamine, pseudoephedrine, brompheniramine, carbinoxamine and their pharmaceutically acceptable salts such as the hydrochloride, maleate, tannate and the like, β-adrenergic receptor antagonists (such as propanolol, nadalol, timolol, pindolol, labetalol, metoprolol, atenolol, esniolol, and acebutolol); narcotic analgesics such as morphine; central nervous system (CNS) stimulants such as methylphenidate hydrochloride; antipsychotics or psychotropics such as phenothiazines, trycyclic antidepressants and MAO inhibitors; benzadiazepines such as alprozolam, diazepam; and the like; and certain non steroidal antinflammatory drugs (NSAIDs), (e.g., salicylates, pyrazolons, indomethacin, sulindac, the fenamates, tolmetin, propionic acid derivatives) such as salicylic acid, aspirin, methyl salicylate, diflunisal, salsalate, phenylbutazone, indomethacin, oxyphenbutazone, apazone, mefenamic acid, meclofenamate sodium, ibuprofen, naproxen, naproxen sodium, fenoprofen, ketoprofen, flurbiprofen, piroxicam, diclofenac, etodolac, ketorolac, aceclofenac, nabumetone, and the like. The drug may be a vasoactive agent, for example, alprostadil for the treatment of erectile dysfunction.

Another pharmacologically active agent useful in this invention is an antigen, i.e., molecule which contains one or more epitopes that will stimulate a host's immune system to make a cellular antigen-specific immune response, or a humoral antibody response. Thus, antigens include proteins, polypeptides, antigenic protein fragments, oligosaccharides, polysaccharides, and the like. The antigen can be derived from any known virus, bacterium, parasite, plants, protozoans, or fungus, and can be a whole organism or immunogenic parts thereof, e.g., cell wall components. An antigen can also be derived from a tumor. An oligonucleotide or polynucleotide which expresses an antigen, such as in DNA immunization applications, is also included in the definition of antigen. Synthetic antigens are also included in the definition of antigen, for example, haptens, polyepitopes, flanking epitopes, and other recombinant or recombinant or synthetically derived antigens (Bergmann et al (1993) Eur. J. Immunol. 23:2777-2781; Bergmann et al (1996) J. Immunol. 157:3242-3249; Suhrbier, A. (1997) Immunol. And Cell Biol. 75:402-408; Gardner et al (1998) 12^{th} World AIDS Conference, Geneva, Switzerland (Jun. 28-Jul. 3, 1998).

Thus when an antigen is associated with gelled alginate in particles in accordance with the invention, it can be viewed as a vaccine composition and as such includes any pharmaceutical composition which contains an antigen and which can be used to prevent or treat a disease or condition in a subject. The term encompasses both subunit vaccines, i.e. vaccine compositions containing antigens which are separate and discrete from a whole organism with which the antigen is associated in nature, as well as compositions containing whole killed, attenuated or inactivated bacteria, viruses, parasites or other microbes. The vaccine can also comprise an adjuvant and/or a cytokine that may further improve the effectiveness of the vaccine.

Viral vaccine compositions used herein included, but are not limited to, those containing, or derived from, members of the families Picornaviridae (e.g., polioviruses, etc.); Caliciviridae; Togaviridae (e.g., rubella virus, dengue virus, etc.); Flaviviridae; Coronaviridae; Reoviridae; Birnaviridae; Rhabodoviridae (e.g., rabies virus, meals virus, respiratory syncytial virus, etc.); Orthomyxoviridae (e.g., influenza virus types A, B and C, etc.); Bunyaviridae; Arenaviridae; Retroviradae (e.g., HTLV-I; HTLV-II; HIV-1; and HIV-2); simian immunodeficiency virus (SIV) among others. Additionally, viral antigens may be derived from a papilloma virus (e.g., HPV); a herpes virus; a hepatitis virus, e.g., (HPV); a herpes virus; a hepatitis virus, e.g., hepatitis A virus (HAV), hepatitis B virus (HBV), hepatitis C virus (HCV), the delta hepatitis D virus (HDV), hepatitis E virus (HEV) AND hepatitis G virus (HGV) and the tick-borne encephalitis viruses. See e.g., Virology, 3^{rd} Edition (W. K. Joklik ed. 1988); Fundamental Virology, 2^{nd} Edition (B. N. Fields and D. M. Knipe, eds. 1991), for a description of these and other viruses. Bacterial vaccine compositions used herein include, but are not limited to, those containing or derived from organisms that cause diphtheria, cholera, tuberculosis, tetanus, pertussis, meningitis, and other pathogenic states, including Meningococcus A, B and C, Hemophilus influenza type B (HIB), and Helicobacter pylori. Examples of anti-parasitic vaccine compositions include those derived from organisms causing malaria and Lyme disease.

Suitable nucleotide sequences for use in the present invention include any therapeutically relevant nucleotide sequence. Thus, the present invention can be used to deliver one or more genes encoding a protein defective or missing from a target cell genome or one or more genes that encode a non-native protein having a desired biological or therapeutic effect (e.g., an antiviral function). The invention can also be used to deliver a nucleotide sequence capable of providing immunity, for example an immunogenic sequence that serves to elicit a humoral and/or cellular response in a subject, or a sequence that corresponds to a molecule having an antisense or ribozyme function.

Suitable genes which can be delivered include those used for the treatment of inflammatory diseases, autoimmune, chronic and infectious diseases, including such disorders as AIDS, cancer, neurological diseases, cardiovascular disease, hypercholestemia; various blood disorders including various anemias, thalassemia and hemophilia; genetic defects such as cystic fibrosis, Gaucher's Disease, adenosine deaminase (ADA) deficiency, emphysema, etc. A number of antisense oligonucleotides (e.g., short oligonucleotides complementary to sequences around the translational initiation site (AUG codon) of an mRNA) that are useful in antisense therapy for cancer and for viral diseases have been described in the art. See, e.g., Han et al 1991) Proc. Natl. Acad. Sci. USA 88:4313; Uhlmann et al (1990) Chem. Rev. 90:543; Helene et al (1990) Biochim. Biophys. Acta. 1049:99; Agarwal et al (1988) Proc. Natl. Acad. Sci. USA 85:7079; and Heikkila et al (1987) Nature 328:445. A number of ribozymes suitable for use herein have also been described. See, e.g., Chec et al (1992) J. Biol. Chem. 267:17479 and U.S. Pat. No. 5,225,347 to Goldberg et al.

For example, in methods for the treatment of solid tumors, genes encoding toxic peptides (i.e., chemotherapeutic agents such as ricin, diphtheria toxin and cobra venom factor), tumor suppressor genes such as p53, genes coding for mRNA sequences which are antisense to transforming oncogenes, antineoplastic peptides such as tumor necrosis factor (TNF) and other cytokines, or transdominant negative mutants of transforming oncogenes, can be delivered for expression at or near the tumor site.

Similarly, genes coding for peptides known to display antiviral and/or antibacterial activity, or stimulate the host's immune system, can also be administered. Thus, genes encoding many of the various cytokines (or functional fragments thereof), such as the interleukins, interferons and colony stimulating factors, will find use with the instant invention. The gene sequences for a number of these substances are known.

For the treatment of genetic disorders, functional genes corresponding to genes known to be deficient in the particular disorder can be administered to the subject. The instant invention will also find use in antisense therapy, e.g., for the delivery of oligonucleotides able to hybridize to specific complementary sequences thereby inhibiting the transcription and/or translation of these sequences. Thus DNA or RNA coding for proteins necessary for the progress of a particular disease can be targeted, thereby disrupting the disease process. Antisense therapy, and numerous oligonucleotides which are capable of binding specifically and predictably to certain nucleic acid target sequences in order to inhibit or modulate the expression of disease-causing genes are known and readily available to the skilled practitioner. Uhlmann et al. (1990) Chem Rev. 90:543, Neckers et al. (1992) Crit. Rev. Oncogenesis 3:175; Simons et al. (1992) Nature 359:67; Bayever et al. (1992) Antisense Res. Dev. 2:109; Whitesell et al. (1991) Antisense Res. Dev. 1:343; Cook et al. (1991) Anti-cancer Drug Design 6:585; Eguchi et al. (1991) Ann. Rev. Biochem. 60:631. Accordingly, antisense oligonucleotides capable of selectively binding to target sequences in host cells are provided herein for use in antisense therapeutics.

The particles of the invention have a size appropriate for high-velocity transdermal delivery to a subject, typically across the stratum corneum or a transmucosal membrane. The mean mass aerodynamic diameter (MMAD) of the particles ranges from about 0.1 to about 250 μm. Thus, the MMAD may be from about 10 to about 100 μm, and preferably from about 10 to about 70 μm or from about 20 to about 70 μm. Generally, the particles will have a very tight size distribution, wherein less than 10% by weight of the particles have a diameter that falls outside of a plus/minus range of 5 μm of the MMAD, that is, less than 10% of the particles will have a diameter at least 5 μm greater than the MMAD or at least 5 μm less than the MMAD. Preferably, no more than 5% by weight of the particles have a diameter which is greater than the MMAD by 5 μm or more. Also preferably, no more than 5% by weight of the particles have a diameter which is smaller than the MMAD by 5 μm or more.

The particles have an envelope density ranging from about 0.1 to 2.5 g/cm³, preferably from about 0.8 to about 1.5 g/cm³. While the shape of the individual particles may vary when viewed under a microscope, the particles are preferably substantially spherical. The average aspect ratio (the ratio of the major axis:minor axis) is typically from 3:1 to 1:1, for example from 2:1 to 1:1. Preferable, the aspect ratio is 1:1 and the particles are thus substantially spherical.

The amount of active agent can be easily varied depending on requirements. The active agent can thus be present in particles in amounts ranging from about 0.1 wt % to about 90 wt % and higher, such as up to 95 wt %. Typically, however the active agent will typically be present in the particles in an amount ranging from about 0.3 wt % to about 85 wt % such as from about 10 wt % to 60 wt % or from about 20 wt % to about 60 wt %. The actual amount depends upon factors such as the activity of the agent and the dose desired.

The rate at which the active agent is released from particles can be altered. Particles with sustained or delayed release capabilities can be provided. Hydrophobic or amphipathic agents can thus be incorporated into the particles in order to slow the hydration rate of dry particles and/or slow the rate of active release from the particles. Such agents can be used to coat the outside of the particles in order to acheive a similar effect. A hydrophilic addictive can be used to hasten release of the active agent.

Examples of hydrophobic agents capable of slowing the hydration and dissolution kinetics of the alginate particles are fatty acids and pharmaceutically acceptable salts thereof (e.g. magnesium stearate, steric acid, zinc stearate, palmitic acid, and sodium palmitate). Other suitable agents include amphiphilic surfactants (glycerides, etc.) or polymers (e.g. polyvinylpyrrolidones (PVPs), derivatised polyethylene glycols (PEGs), etc). Starch components are also suitable for these purposes, as are semi-miscible solvents (i.e. solvents with partial miscibility in water) such as triacetin which can be added to the particles to serve as a dissolution barrier.

These solvents can further be employed to load hydrophobic agents into the alginate particles that otherwise would not readily pass into the hydrophilic environment of the algiante structure. In order to incorporate such agents into the particles, suitable methods can employ an organic or alcohol base solvent in which the hydrophobic agent is dissolved. Dry particles can then be placed within the solvent in a ratio of solvent to particles that is at least sufficient to wet completely the particle surface. Additional solvent can then be used to build up thicker coatings. The final amount of hydrophobic agent absorbed into or onto the particles depends upon the concentration of the coating agent in the solvent, the degree of swelling experienced by the particles in the solvent and the relative amounts of the solvent and particles.

If required, the alginate particles can be further strengthened to improve their suitability for, and performance in high velocity particle injection methods. The particles may comprise chitosan for this purpose. Alternatively, the particles may be coated with a polycation. Suitable polycations include poly-L-lysine and polyvinylamine. The particles may further comprise a locally acting active agent such as a vasodilator or vasoconstrictor, epinephrine or methylnicotine.

As noted above, the particles are typically provided in the form of a free-flowing powder. The powder may also include one or more pharmaceutically acceptable excipient such as a binder, carrier, stabilizer, glidant, antioxidant, pH adjuster, anti-irritant and the like. Such an excipient generally refers to a substantially inert material that is nontoxic and does not interact with other components in a deleterious manner. The proportions in which a particular excipient may be present depend upon the purpose for which the excipient is provided and the identity of the excipient. Carriers or diluents such as dextran may be provided in any suitable amount such as from about 10 to about 75% by weight of the particles, for example from about 20 to about 70%, or from about 30 to about 60% by weight.

Examples of suitable carrier excipients that also act as stabilizers for peptides include pharmaceutical grades of dextrose, sucrose, lactose, trehalose, mannitol, sorbitol, inositol, dextran and the like. The carrier may thus be a saccharide such as a monosaccharide, a disaccharide or a sugar alcohol. Other carriers include starch, cellulose, sodium or calcium phosphates, calcium sulfate, citric acid, tartaric acid, glycine, high molecular weight polyethylene glycols (PEG), and combinations thereof. It may also be useful to employ a charged lipid and/or detergent. Suitable charged lipids include, without limitation, phosphatidylcholines (lecithin), and the like. Detergents will typically be a nonionic, anionic, cationic or amphoteric surfactant. Examples of suitable surfactants include, for example, Tergitol® and Triton® surfactants (Union Carbide Chemicals and Plastics, Danbury, Conn.), polyoxyethylenesorbitans, e.g., TWEEN® surfactants (Atlas Chemical Industries, Wilmington, Del.), polyoxyethylene ethers, e.g. Brij, pharmaceutically acceptable fatty acid esters, e.g., lauryl sulfate and salts thereof (SDS), and like materials.

It may also be useful to use a penetration enhancer for the skin to assist in the delivery profile of particles. A “penetration enhancer” or “permeation enhancer” as used herein relates to an agent which increases the permeability of skin to a pharmacologically active agent, i.e. so as to increase the rate at with the agent permeates through the skin and enters the bloodstream. The enhanced permeation effected through the use of such enhancers can be observed by measuring the rate of diffusion of an active agent through animal or human skin using a diffusion cell apparatus well known in the art.

Alginate particles loaded with a pharmacologically active agent can be prepared by a process comprising:

(a) providing an aqueous solution or dispersion of the pharmacologically active agent, within which solution or dispersion a water-soluble alginate is dissolved;

(b) mixing the aqueous solution or dispersion with a sufficient amount of a water-immiscible solvent so as to form an emulsion in which droplets of the aqueous solution or dispersion are dispersed in the water-immiscible solvent;

(c) adding a divalent or trivalent metal cation which gels the alginate;

(d) collecting the resultant gelled alginate particles loaded with the pharmacologically active agent; and

(e) if necessary, separating therefrom the required alginate particles.

The alginate facilitates the particle-forming process. It acts as a viscosity enhancer for the dispersed phase, i.e. for the aqueous solution or dispersion of the pharmacologically active agent that is dispersed in the water-immiscible solvent. The alginate thus acts to equalise the viscosity difference between the dispersed phase and the continuous phase, thereby enabling a stable emulsion to form. The alginate also acts as a cross-linking binder, thereby enabling solidification of the emulsified droplets after appropriate droplet sizes have been established.

The water-soluble alginate in step (a) may be a sodium, potassium, magnesium or ammonium alginate. Sodium alginate is preferred. The alginate may be formed in situ by addition of an alkali such as NaOH to a solution of alginic acid. The sodium alginate may be a low, medium or high viscosity sodium alginate. The viscosity of sodium alginate is determined by reference to a 2% by weight solution at 25° C. Under such conditions, a low viscosity sodium alginate has a viscosity of about 250 cps, a medium viscosity sodium alginate has a viscosity of about 3,500 cps and a high viscosity sodium alginate has a viscosity of about 14,000 cps.

The concentration of the water-soluble alginate and pharmacologically active agent in the aqueous solution/dispersion of step (a) can be selected as desired. The pH can be adjusted to maximise solubility. A water-soluble chitosan such as chitosan hydrochloride can be included in the aqueous solution/dispersion.

The water-immiscible solvent in step (b) may be an oil, such as a light vegetable oil or a light mineral oil. The solvent may be a fatty alcohol such as an alkanol, for example a C₇-C₁₅ alkanol such as octanol. Preferably the solvent is decalin (decahydronaphthalene). An emulsion forms. Generally, a stable emulsion is formed. The water-immiscible solvent is present in excess and constitutes the continuous phase of the emulsion. Droplets of the aqueous solution constitute the dispersed phase. Stirring is typically carried out continuously. The conditions, particularly the stirring, can be controlled so that droplets of the aqueous solution/dispersion are formed having the size desired for gelation.

A divalent or trivalent metal cation that is capable of gelling alginate is added in step (c). Typically, an aqueous solution of a salt of the metal cation is added. Depending upon the metal cation, the water soluble salt may be a chloride, gluconate, lactate, acetate or sulfate salt. A solution of calcium chloride, gluconate or lactate or a solution of zinc acetate, sulfate, chloride or gluconate may be employed for this purpose. A solution of calcium chloride is preferred. More than one metal cation may be added. For example, calcium chloride and zinc chloride solution(s) may be added. The stirring is continued in step (c) and the alginate gels, thereby to form particles loaded with the pharmacologically active agent.

In step (d), the alginate particles can be filtered off. They may be washed, for example with an alcohol such as isopropyl alcohol. They can then be dried. Any suitable drying method can be used, for example spray-drying, freeze-drying, spray-freeze drying, air-drying, vacuum-assisted drying, fluid bed drying and the like. However, vacuum-assisted drying, fluid bed drying, and freeze-drying are preferred.

The particles may be sieved in step (e) to remove particles having the wrong size. The resulting particles have suitable physical and functional characteristics for direct injection by a needleless syringe.

Alginate particles loaded with a pharmacologically active agent may alternatively be formed by a process comprising:

(a) providing pre-formed alginate particles which are not loaded with the pharmacologically active agent;

(b) contacting the particles with an aqueous solution or dispersion of the pharmacologically active agent for a period of time sufficient to allow the particles to swell and incorporate the active agent therewithin;

(c) collecting the particles thus loaded with the pharmacologically active agent; and

(d) if necessary, separating therefrom the require alginate particles.

Blank alginate particles, i.e. particles which are not loaded with the pharmacologically active agent, are provided in step (a). These are generally prepared with the characteristics, for example size and density, required for needleless injection. Typically, the blank particles are suspended in the aqueous solution or disperion of the pharmacologically active agent in step (b). The particles collected in step (c) can be washed and/or dried as necessary. They may be sieved in step (d) to obtain particles having the required size.

The particles collected in step (c) or (d) can be recycled to step (b). Steps (b), (c) and optionally (d) may thus be carried out several times, for example from 2 to 10 times, to increase each time the amount of active agent that is loaded in the particles. The particles may be partially or completely dried for such recycling. Again, any suitable drying method can be used. Partial drying can be effected using a solvent such as acetone or an air-drying method. For the final drying step, vacuum-assisted drying, fluid bed drying, and freeze-drying are again preferred.

The alginate particles are used for delivering a pharmacologically active agent to a subject in need thereof. The alginate particles are administered by means of a needleless injection. Direct transdermal delivery may be adopted, typically across a skin surface (e.g. through the stratum corneum) or into a mucosal membrane.

The alginate particles are accelerated to a high velocity and delivered to a target surface present on a subject. Injection velocities generally range from 100 to 3000 m/sec such as from 200 to 2000 m/sec. The target surface is typically a predetermined area of intact unbroken living skin or mucosal tissue. That area will usually be in the range of about 0.3 cm² to about 10 cm².

The pharmacologically active agent may be delivered for any desired purpose. Thus, the active agent may be administered for diagnosis, treatment or prevention of a condition in a subject. As used herein, the terms “treatment” and “treating” include any of the following: the prevention of infection or reinfection; the reduction or elimination of symptoms; and the reduction or complete elimination of a pathogen. Treatment may be effected prophylactically (prior to infection) or therapeutically (following infection).

By using a needleless syringe, a therapeutically effective amount of the pharmacologically active agent can be delivered to the subject. A therapeutically effective amount is that amount needed to give the desired pharmacologic effect. This amount will vary with the relative activity of the agent to be delivered and can be readily determined through clinical testing based on known activities of the compound being delivered. The “Physicians Desk Reference” and “Goodman and Gilman's The Phamacological Basis of Therapeutics” are useful for the purpose of determining the amount needed.

Needleless syringe devices for delivering particles were first described in commonly owned U.S. Pat. No. 5,630,796 to Bellhouse et al, incorporated herein by reference. Although a number of specific device configurations are now available, such devices are typically provided as a pen-shaped instrument containing, in linear order moving from top to bottom, a gas cylinder, a particle cassette or package, and a supersonic nozzle with an associated silencer medium. An appropriate powder (in the present case, a powder comprising the alginate particles loaded with the pharmacologically active agent) is provided within a suitable container, e.g. a cassette formed by two rupturable polymer membranes that are heat-sealed to a washer-shaped spacer to form a self-contained sealed unit. Membrane materials can be selected to achieve a specific mode of opening and burst pressure that dictate the conditions at which the supersonic flow is initiated.

In operation, the device is actuated to release the compressed gas from the cylinder into an expansion chamber within the device. The released gas contacts the particle cassette and, when sufficient pressure is built up, suddenly breaches the cassette membranes sweeping the particles into the supersonic nozzle for subsequent delivery. The nozzle is designed to achieve a specific gas velocity and flow pattern to deliver a quantity of particles to a target surface of predefined area. The silencer is used to attenuate the noise produced by the supersonic gas flow.

A second needleless syringe device for delivering particles is described in commonly owned International Publication No. WO 96/20022. This delivery system also uses the energy of a compressed gas source to accelerate and deliver powdered compositions. However, it is distinguished from the system of U.S. Pat. No. 5,630,796 in its use of a shock wave instead of gas flow to accelerate the particles. More particularly, an instantaneous pressure rise provided by a shock wave generated behind a flexible dome strikes the back of the dome, causing a sudden eversion of the flexible dome in the direction of a target surface. This sudden eversion catapults a powdered composition (which is located on the outside of the dome) at a sufficient velocity, thus momentum, to penetrate target tissue, e.g., oral mucosal tissue. The powdered composition is released at the point of full dome eversion. The dome also serves to completely contain the high-pressure gas flow which therefore does not come into contact with the tissue. Because the gas is not released during this delivery operation, the system is inherently quiet. This design can be used in other enclosed or otherwise sensitive applications for example, to deliver particles to minimally invasive surgical sites.

Suitable needleless syringe devices are the dermal PowderJect® needleless syringe and the oral PowderJect® needleless syringe (PowderJect Technologies Limited, Oxford, UK).

Single unit dosages or multidose receptacles, in which the alginate particles of the invention may be packaged or otherwise contained prior to use, can comprise a hermetically sealed container enclosing a suitable amount of the particles that make up a suitable dose. The particle compositions can be packaged as a sterile formulation, and the hermetically sealed container can thus be designed to preserve sterility of the formulation until use. These receptacles can be adapted for direct use in a particular type of needleless syringe.

Particles of the present invention can thus be packaged in individual unit dosages for delivery via any needleless syringe, for example those needleless syringes referenced herein above. As used herein, a “unit dosage” intends a single dosage receptacle containing a therapeutically effective amount of a powder of the invention. The unit dosage receptacle typically fits within a needleless syringe device to allow for transdermal delivery from the device. Such receptacles can be capsules, foil pouches, sachets, cassettes or the like.

More particularly, the unit dosage receptacle will typically be comprised of two rupturable diaphragms or membranes that are sealed or otherwise connected together about their edges to form a common sachet, capsule or other sealed unit containing a dose of the alginate particles of the present invention. These diaphragms and/or membranes can be sealed or otherwise connected directly or indirectly, for example, sealed, adhered or otherwise affixed to opposite axial faces of an intervening ring.

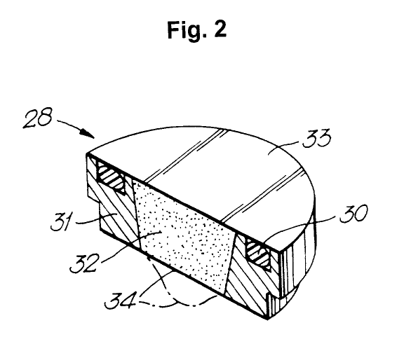

An exemplary unit dosage receptacle according to the present invention is depicted in FIG. 2. As shown in that Figure, the unit dosage receptacle 28 can comprise an annular ring 31, having a substantially cylindrical or even a frustoconical (as shown) internal periphery defining a compartment 32 containing a dosage of the alginate particles of the present invention. The top of the compartment is closed off by an upper diaphragm or membrane 33 and at the bottom by a lower diaphragm or membrane 34. The diaphragms or membranes 33 and 34 can be formed from any suitable rupturing material, wherein the rupture or burst character is controlled by the selection of material and the physical dimension of such material, for example thickness. The diaphragms or membranes 33 and 34 can be sealed to the upper and lower walls, respectively, of the ring 31 by compression fit within a needleless syringe, e.g., by compression between two parts of such a syringe, but are preferably heat or otherwise adhered or bonded to the faces of the ring such that a self-contained, hermetically sealed unit dosage receptacle is thereby formed. In addition, an optional O-ring 30 can be provided in a recess formed in the upper wall of the annular ring 31, thus providing for a better seal or fit once placed within a suitable needleless syringe. Furthermore, the unit dosage receptacle may optionally include three or more diaphragms and/or membranes, for example to split the compartment 32 into separate upper and lower portions, and thus house isolated particle dosages that are kept separate and isolated from each other until delivery from the needleless syringe.

The unit dosage receptacle in which the particles are packaged or otherwise contained can further be labeled to identify the composition and provide relevant dosage information. In addition, the receptacle can be labelled with a notice in the form prescribed by a governmental agency, for example the Food and Drug Administration, wherein the notice indicates approval by the agency under Federal law of the manufacture, use or sale of the alginate compositions contained therein for human administration.

If desired, needleless syringes can be provided in a preloaded condition containing a suitable unit dosage receptacle containing the alginate particles of the invention. The loaded syringe can be packaged in a hermetically sealed container, which may further be labeled as described above.

A number of test methods have been developed or modified in order to characterize performance of particles administered using a needleless syringe device. These tests range from characterization of the powdered composition, assessment of the gas flow and particle acceleration, impact on artificial or biological targets, and measures of complete system performance. One, several or all of the following tests can thus be employed to assess the physical and functional suitability of the present alginate particles for use in a needleless syringe system.

Assessment of Effect on Artificial Film Targets

A functional test that measures many aspects of powder injection systems simultaneously has been designated as the “metallized film” or “penetration energy” (PE) test. It is based upon the quantitative assessment of the damage that particles can do to a precision thin metal layer supported by a plastic film substrate. Damage correlates to the kinetic energy and certain other characteristics of the particles. The higher the response from the test (i.e., the higher the film damage/disruption) the more energy the device has imparted to the particles. Either electrical resistance change measurement or imaging densitometry, in reflectance or transmission mode, provide a reliable method to assess device or formulation performance in a controllable and reproducible test.

The film test-bed has been shown to be sensitive to particle delivery variations of all major device parameters including pressure, dose, particle size distribution and material, etc. and to be insensitive to the gas. Aluminum of about 350 Angstrom thickness on a 125 μm polyester support is currently used to test devices operated at up to 60 bar.

Assessment of Impact Effect on Engineering Foam Targets

Another means of assessing particle performance when delivered via a needleless syringe device is to gauge the effect of impact on a rigid polymethylimide foam (Rohacell 5 IIG, density 52 kg/m³, Rohm Tech Inc., Malden, Mass.). The experimental set-up for this test is similar to that used in the metallized film test. The depth of penetration is measured using precision calipers. For each experiment a processed mannitol standard is run as comparison and all other parameters such as device pressure, particle size range, etc., are held constant. Data also show this method to be sensitive to differences in particle size and pressure. Processed mannitol standard as an excipient for drugs has been proven to deliver systemic concentrations in preclinical experiments, so the relative performance measure in the foam penetration test has a practical in vivo foundation. Promising powders can be expected to show equivalent or better penetration to mannitol for anticipation of adequate performance in preclinical or clinical studies. This simple, rapid test has value as a relative method of evaluation of powders and is not intended to be considered in isolation.

Particle Attrition Test

A further indicator of particle performance is to test the ability of various candidate compositions to withstand the forces associated with high-velocity particle injection techniques, that is, the forces from contacting particles at rest with a sudden, high velocity gas flow, the forces resulting from particle-to-particle impact as the powder travels through the needleless syringe, and the forces resulting from particle-to-device collisions also as the powder travels through the device. Accordingly, a simple particle attrition test has been devised which measures the change in particle size distribution between the initial composition, and the composition after having been delivered from a needleless syringe device.

The test is conducted by loading a particle composition into a needleless syringe as described above, and then discharging the device into a flask containing a carrier fluid in which the particular composition is not soluble (e.g. mineral oil, silicone oil, etc.). The carrier fluid is then collected, and particle size distribution in both the initial composition and the discharged composition is calculated using a suitable particle sizing apparatus, e.g., an AccuSizer® model 780 Optical Particle Sizer. Compositions that demonstrate less than about 50%, more preferably less than about 20% or about 10%, reduction in mean mass aerodynamic diameter (as determined by the AccuSizer apparatus) after device actuation are deemed suitable for use in the needleless syringe systems described herein.

Delivery to Human Skin in vitro and Transepidermal Water Loss

For a composition performance test that more closely parallels eventual practical use, candidate particle compositions can be injected into dermatomed, full thickness human skin samples. Replicate skin samples after injection can be placed on modified Franz diffusion cells containing 32° C. water, physiologic saline or buffer. Additives such as surfactants may be used to prevent binding to diffusion cell components and to maintain sink conditions. Two kinds of measurements can be made to assess performance of the formulation in the skin.

To measure physical effects, i.e. the effect of particle injection on the barrier function of skin, the transepidermal water loss (TEWL) can be measured. Measurement is performed at equilibrium (about 1 hour) using a Tewameter TM 210® (Courage & Khazaka, Koln, Ger) placed on the top of the diffusion cell cap that acts like a ˜12 mm chimney. Larger particles and higher injection pressures generate proportionally higher TEWL values in vitro and this has been shown to correlate with results in vivo. Upon particle injection in vitro TEWL values increased from about 7 to about 27 (g/m²h) depending on particle size and helium gas pressure. Helium injection without powder has no effect. In vivo, the skin barrier properties return rapidly to normal as indicated by the TEWL returning to pretreatment values in about 1 hour for most powder sizes. For the largest particles, 53-75 μm, skin samples show 50% recovery in an hour and full recovery by 24 hours.

Delivery to Human Skin in vitro and Drug Diffusion Rate

To measure the formulation performance in vitro, the active (guest) component(s) of candidate compositions can be collected by complete or aliquot replacement of the Franz cell receiver solution at predetermined time intervals for chemical assay using HPLC or other suitable analytical technique. Concentration data can be used to generate a delivery profile and calculate a steady state permeation rate. This technique can be used to screen formulations for early indication of drug binding to skin, drug dissolution, efficiency of particle penetration of stratum corneum, etc., prior to in vivo studies.

These and other qualatative and quantitative tests can be used to assess the physical and functional suitability of the present particles for use in a high-velocity particle injection device. It is preferred, though not required, that the particles have the following characteristics: a substantially spherical shape (e.g. an aspect ratio as close as possible to 1:1 such as from 1.5:1); a smooth surface; have a high active agent loading content (e.g. up to 80 or 90% loading); less than 20% reduction in particle size using the particle attrition test; an envelope density as close as possible to the true density of the constituents (e.g. greater than about 0.8 g/ml); and a MMAD of about 20 to 70 μm with a narrow particle size distribution. The compositions may be free-flowing (e.g. free flowing after 8 hours storage at 50% relative humidity and after 24 hours storage at 40% relative humidity). All of these criteria can be assessed using the above-described methods, and are further detailed in the following publications, incorporated herein by reference. Etzler et al (1995) Part. Part. Syst. Charact. 12:217; Ghadiri, et al (1992) IFPRI Final Report, FRR 16-03 University of Surrey, UK; Bellhouse et al (1997) “Needleless delivery of drugs in dry powder form, using shock waves and supersonic gas flow,” Plenary Lecture 6, 21^{st}International Symposium on Shock Waves, Australia; and Kwon et al (1998) Pharm. Sci. suppl. 1 (1), 103.

C. Experimental

Below are examples of specific embodiments for carrying out the methods of the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### EXAMPLE 1

### Preparation of Alginate Particles Loaded with Lysozyme

A 1% by weight alginate solution was prepared by dissolving 0.203 g of alginic acid (Spectrum Quality Products) in 20 g of distilled water and adding four drops of 1N NaOH to obtain a clear solution. In 5 g of the resulting solution, 0.33 g lysozyme was dissolved (to obtain an approximately 86% lysozyme solids content). The lysozyme-containing solution was then mixed with 25 g of decalin and homogenised. A liquid-liquid emulsion formed in which the decalin constituted the continuous phase and the lysozyme-containing solution constituted the discontinuous phase.

As the mixing continued, 5 ml of a CaCl₂ was added to the emulsion when the desired degree of homogenisation had been reached. Gelation of the alginate occurred and particles of alginate, within which lysozyme was entrapped, formed. These particles were filtered off, washed with ethanol and freeze-dried. The resultant product was comprised of a free-flowing powder of generally spherical particles.

### EXAMPLE 2

### Investigation of Transdermal Flux

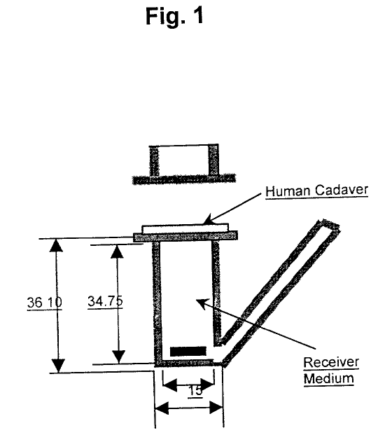

Modified Franz cells (6.9 ml) were designed for investigation of transdermal flux (FIG. 1). The receiver medium was phosphate buffered saline (PBS) with NaOH and to which 0.5% Tween 80 (w/v) had been added to minimise lysozyme adsorption to glass, and to maintain infinite sink conditions. This medium was demonstrated to have no effect on lysozyme permeability of dermatomed skin. The temperature of the receiver was maintained at 32° C. during the experiments. Full thickness (about 300 μm) skin from human cadavers was used. Sieved fractions of neat lysozyme particles having a size of 38-53 μm were prepared by lyophilisation, compression and milling. Alginate particles loaded with lysozyme were prepared according to the procedure described in Example 1 and sieved fractions 38-53 μm in size were collected. A total of 3 mg of each type of particle formulation were injected to the cell using a dermal PowderJect® ND1 needleless syringe device (PowderJect Technologies Ltd., Oxford UK) operated at 60 bar of helium gas. At predetermined times, samples were assayed for lysozyme content by high pressure liquid chromatography HPLC). The results are shown in the Table below.

TABLE
Summary of cumulative amounts over 24 hours

| | | Delivery Amt. | Bioavailability |
|---|---|---|---|
| Formulation | Size (μm) | (μg/24 hrs) | (%) |
| Neat lysozyme | 38-53 | 213 ± 178 | 7.1 |
| Alginate | 38-53 | 249 ± 170 | 63.8 |

All publications and patent applications mentioned in this specification are herein incorporated by reference to the same extent as if each individual publication or patent application was specifically and individually indicated to be incorporated by reference.

The invention now being fully described, it will be apparent to one or ordinary skill in the art that many changes and modifications can be made thereto without departing from the spirit or scope of the appended.

What is claimed is:

1. A method of delivering a pharmacologically active agent to a subject, which method comprises the step of administering to the subject by needleless injection an effective amount of alginate particles which are loaded with the said agent and which have a mean mass aerodynamic diameter of from 0.1 to 250 μm and an envelope density of from 0.1 to 2.5 g/cm³.

2. A method according to claim 1, wherein the mean mass aerodynamic diameter of the particles is from 10 to 70 μm, less than 10% by weight of the particles have a diameter which is at least 5 μm less or greater than the mean mass aerodynamic diameter of the said particles, the envelope density of the particles ranges from 0.8 to 1.5 g/cm³, and the aspect ratio of the particles ranges from 3:1 to 1:1.

3. A method according to claim 1 wherein the reduction in the mean mass aerodynamic diameter of the particles in the Particle Attrition Test is less than 20%.

4. A method according to claim 1 wherein the pharmacologically active agent is a protein, peptide, nucleic acid or vaccine.

5. A method according to claim 1 wherein the alginate is calcium alginate.

6. A method according to claim 1 wherein the alginate is composed of 25 to 35 wt % guluronate residues and 75 to 65 wt % mannuronate residues.

7. A method according to claim 1 wherein the alginate is composed of 35 to 60 wt % guluronate residues and 65 to 40% mannuronate residues.

8. A method according to claim 1 wherein the alginate is composed of 60 to 70 wt % guluronate residues and 40 to 30% mannuronate residues.

9. A method according to claim 1 wherein the particles incorporate chitosan.

10. A method according to claim 1 wherein the particles are coated with a polycation.

11. A unit dosage receptable for use a needleless syringe, said receptacle containing a dose of alginate particles which are loaded with a pharmacologically active agent, wherein said particles have a mean mass aerodynamic diameter of from 0.1 to 250 μm and an envelope density of from 0.1 to 2.5 g/cm³.

12. A receptacle according to claim 11 wherein the mean mass aerodynamic diameter of the particles is from 10 to 70 μm, less than 10% by weight of the particles have a diameter which is at least 5 μm less or greater than the mean mass aerodynamic diameter of the said particles, the envelope density of the particles is from 0.8 to 1.5 g/cm³, and the aspect ratio of the particles ranges from 3:1 to 1:1.

13. A receptacle according to claim 11 wherein the reduction in the mean mass aerodynamic diameter of the particles in the Particle Attrition Test is less than 20%.

14. A receptacle according to claim 11 wherein the pharmacologically active agent is a protein, peptide, nucleic acid or vaccine.

15. A receptacle according to claim 11 wherein the alginate is calcium alginate.

16. A receptacle according to claim 11 wherein the alginate is composed of 25 to 35 wt % guluronate residues and 75 to 65 wt % mannuronate residues.

17. A receptacle according to claim 11 wherein the alginate is composed of 35 to 60 wt % guluronate residues and 65 to 40% mannuronate residues.

18. A receptacle according to claim 11 wherein the alginate is composed of 60 to 70 wt % guluronate residues and 40 to 30% mannuronate residues.

19. A receptacle according to claim 11 wherein the particles incorporate chitosan.

20. A receptacle according to claim 11 wherein the particles are coated with a polycation.

21. A receptacle according to claim 11 which is selected from the group consisting of capsules, foil pouches, sachets and cassettes.

22. Alginate particles suitable for administration to a subject by needleless injection, wherein the particles are loaded with a pharmacologically active agent, the mean mass aerodynamic diameter of the particles is from 10 to 100 μm, less then 10% by weight of the particles have a diameter which is at least 5 μm less or greater than the mean mass aerodynamic diameter of the said particles, the envelope density of the particles is from 0.8 to 1.5 g/cm³, and the aspect ratio of the particles ranges from 3:1 to 1:1.

23. Particles according to claim 22 wherein the reduction in the mass mean aerodynamic diameter of the particles in the Particle Attrition Test is less than 20%.

24. Particles according to claim 22 wherein the pharmacologically active agent is a protein, peptide, nucleic acid or vaccine.

25. Particles according to claim 22 wherein the alginate is calcium alginate.

26. Particles according to claim 22 wherein the alginate is composed of 25 to 35 wt % guluronate residues and 75 to 65 wt % mannuronate residues.

27. Particles according to claim 22 wherein the alginate is composed of 35 to 60 wt % guluronate residues and 65 to 40% mannuronate residues.

28. Particles according to claim 22 wherein the alginate is composed of 60 to 70 wt % guluronate residues and 40 to 30% mannuronate residues.

29. Particles according to claim 22 wherein the particles incorporate chitosan.

30. Particles according to claim 22 where the particles are coated with a polycation.

31. A process for the preparation of alginate particles suitable for administration to a subject by needleless injection wherein the particles are loaded with a pharmacologically active agent, the mean mass aerodynamic diameter of the particles is from 10 to 100 μm, less than 10% by weight of the particles have a diameter which is at least 5 μm less or greater than the mean mass aerodynamic diameter of the said particles, the envelope density of the particles is from 0.8 to 1.5 g/cm³, and the aspect ratio of the particles ranges from 3:1 to 1:1; which process comprises the steps of:
(a) providing an aqueous solution or dispersion of the pharmacologically active agent, within which solution or dispersion a water-soluble alginate is dissolved;
(b) mixing the aqueous solution or dispersion with a sufficient amount of a water-immiscible solvent so as to form an emulsion in which droplets of the aqueous solution or dispersion are dispersed in the water-immiscible solvent;
(c) adding a divalent or trivalent metal cation which gels the alginate; and
(d) collecting the resultant gelled alginate particles loaded with the pharmacologically active agent.

32. A process for the preparation of alginate particles suitable for use in a needleless injection, wherein the particles are loaded with a pharmacologically active agent, the mean mass aerodynamic diameter of the particles is from 10 to 100 μm, less then 10% by weight of the particles have a diameter which is at least 5 μm less or greater than the mean mass aerodynamic diameter of the said particles, the envelope density of the particles is from 0.8 to 1.5 g/cm³, and the aspect ratio of the particles ranges from 3:1 to 1:1; which process comprises the steps of:
(a) providing pre-formed alginate particles which are not loaded with the pharmacologically active agent;
(b) contacting the particles with an aqueous solution or dispersion of the pharmacologically active agent for a period of time sufficient to allow the particles to swell and incorporate the active agent therewithin; and
(c) collecting the particles thus loaded with the pharmacologically active agent.
